(19) United States
(12) Patent Application Publication
(10) Pub. No.: US 2006/0153855 A1
(43) Pub. Date: Jul. 13, 2006

(54) Prevention and treatment of the porcine reproductive and respiratory syndrome (PRRS)

(76) Inventors: Jose Andres Morales Garzon, Tehuacan (MX); Eduardo Lucio Decanini, Tehuacan (MX)

Correspondence Address: LOCKE LIDDELL & SAPP LLP;ATTN: STACIE COX, 2200 ROSS AVENUE, SUITE 2200, DALLAS, TX, 75201-6776, US

(21) Appl. No.: 11/286,932

(22) Filed: Nov. 23, 2005

Related U.S. Application Data
(63) Continuation-in-part of application No. 10/695,104, filed on Oct. 28, 2003, now abandoned.

(30) Foreign Application Priority Data
Oct. 30, 2002 (MX) PA/A/2002/011761

(51) Int. Cl. A61K 39/42

(52) U.S. Cl. 424/159.1

(57) ABSTRACT

The present invention relates to the use of compositions of specific anti-PRSSV immunoglobulins obtained from the egg yolk of hens hyperimmunized with PRRS virus. The immunoglobulins are obtained through the extraction of the aqueous phase of the yolk through the use of hydroxypropylmethylcellulose phtalate at a final concentration of 0.05% and sodium azide at a final concentration of 0.001%. The invention also relates to the orally and parenterally administration of these immunoglobulins for the prevention and treatment of pigs infected with the PRRS virus in order to lower mortality rates, obtain weight gain and diminish viral excretion in the herds.

### CROSS REFERENCE TO RELATED APPLICATIONS

This non-provisional application is a continuation-in-part of prior application Ser. No. 10/695,104 filed Oct. 23, 2003, which claimed priority to Mexican patent application PA/A/2002/011761 filed Oct. 30, 2002.

### FIELD OF THE INVENTION

The present invention relates to a new method for the treatment and prevention of porcine reproductive and respiratory syndrome (PRRS) based on the parenteral administration of immunoglobulins obtained from egg yolk from hens hyperimmunized with the PRRS virus.

### BACKGROUND OF THE INVENTION

The Porcine Reproductive and Respiratory Syndrome (PRRS) is a serious illness affecting pigs, which was reported in the United States of America in 1987 and was then identified in various European countries. In 1991, Holland reported the isolation of the etiological agent called Lelystad virus and because of the symptoms presented by the pigs, it was known as the Porcine Epidemic Abortion and Respiratory Syndrome.

There are two ways of protecting animals against infectious agents: they can be exposed to antigens derived from an infectious agent to stimulate a protective immunity reaction or they can receive a preformed antibody obtained from an immune subject. The first way is conducted through different types of vaccines: freeze-dried live viruses or bacteria, dead viruses or bacteria in oily emulsions; and recently the creation of cloned and recombinant vaccines. Each of them presents advantages and disadvantages with regard to protection, immune response and protection duration. In some cases, there are also undesirable lesions in the host because of the vaccine virus (Tizard, I. R. 1998).

The second form of protection, also called passive immunity, includes the transmission of antibodies specific against infectious agents in a host.

Traditionally, at the research level, the antibodies are mainly obtained in mammals and less frequently in birds. The types of antibodies obtained are monoclonal and polyclonal antibodies in mammals and polyclonal antibodies in birds (Larsson, et al. 1993).

In the case of birds, the chicken is the only species from which antibodies are obtained in a most accessible and highly defined form. The main serum antibody present in the chicken is IgG even though IgG is transported to the egg in a way similar to the transfer of mammal IgG through the placenta.

In the egg, IgG is found in higher concentrations in the yolk, although it is also found in small concentrations in the albumin; it is even found in larger quantities in the yolk than in the hen serum (Larsson, et al. 1993).

To have an idea of the quantity of antibodies made in the hen, we must take into account that an egg-laying hen produces approximately 5 to 6 eggs per week with a yolk volume of about 15 ml. Thus, in a week, a hen produces antibodies in yolk equivalent to 90-100 ml of serum or 180-200 ml of whole blood. This is to be compared with the 20 ml of whole blood given per week by an immunized rabbit. Obviously if we use animals such as horses or cows, the quantity of serum and antibodies is larger than in the egg but it is more expensive and more painful for the animals.

Among the advantages of the antibodies found in the yolk of hen egg, we can mention the following ones:
1. They do not fix the complement;
2. They do not bind to the Protein A of Staphilococcus aureus;
3. They do not react with the Rheumatoid Factor;
4. Because of its phylogenetic difference with mammal antibodies, the IgG does not cross react with the mammal antibodies; and
5. Low cost.

Recently, egg yolk antibodies (immunoglobulins) have been employed as tools for diagnostic and therapy (Schmidt, et al. 1989). Thus, taking advantage of its phylogenetic difference with mammal immunoglobulins, the Ig's have presented several advantages when used in immune diagnosis. For example, yolk Ig's have been used to detect several viruses through ELISA, immunodiffusion, immunofluorescence and complement fixing. Because of its low isoelectric point, compared to human Ig, they are employed in electrophoresis assays for the quantification of immunoglobulins in the serum of several animals (Altschuh, D. 1984, Larsson, et al. 1988, Larsson, et al. 1992, Larsson, et al. 1993, Schade, R. 1996). With regard to their therapeutic application, the Ig's have been used as immunotherapy in several scientific fields. For example, the administration of egg yolk immunoglobulins orally has prevented rotavirus infections in mice, bovines, and pigs, among others (Ikemori, et al 1992, Kuroki, et al 1994, Marquardt, et al 1998). Moreover, they have been used as antivenoms against viper and scorpions, that can be injected to neutralize the toxins without the risk of anaphylactic reactions commonly caused by antivenoms elaborated in horse (Larsson, et al. 1993). A further application has been to prevent caries caused by Streptococcus mutans in humans (Hatta, H. et al 1984).

Although the process to obtain immunoglobulins from any animal seems very easy, in the field it is very difficult because infections caused by bacteria or virus are very different. Each microorganism has its own mechanism of action and requires a lot of expensive experimentation.

In the PRRS field, a lot of people worked for years to find methods to prevent or treat the virus' infections, but the results have been not as expected.

In the U.S. Pat. No. 6,217,865 Hunchar claims a method to increase efficacy of egg-obtained immunoglobulins by mixing eggs harvested for a period of 30-60 days after immunization but he only experimented with a single vaccine to cholecystokinin.

The National Pork Board is funding at the University of Minnesota a PRRS Database for Sequence Comparison that contains more than 4,000 ORF5 sequences from PRRS isolates, including archived information at PRRS virus sequencing centers. It's open to the public to further the global understanding of PRRS virus and to help solve the problem of PRRS through development of effective vaccines. Opriessnig, V. Et al 2005 referencia completa carried out research that concluded that ORF5 sequences between a modified PRRS virus vaccine and DNA sequence of PRRS virus found in farms couldn't predict effectiveness of the vaccine.

### SUMMARY OF THE INVENTION

The object of the present invention is to offer a method for prevention and treatment of infection caused by PRRS virus through the parenteral and oral administration of immunoglobulins, obtained from the egg yolk of hens hyperimmunized with one or several PRRS viruses.

Another object of the present invention is to foment weight increase in animals treated with immunoglobulins specifically directed against PRRS virus.

Moreover, within the present invention, the use of immunoglobulins obtained form egg yolk against PRRS virus is claimed to eliminate or substantially reduce the signology and mortality, transmission and prevention of PRRS virus in treated animals.

Finally, the invention relates to a process to prepare a product based on immunoglobulins obtained from egg yolk specifically directed against PRRS.

Through the practice of the present invention, the dissemination of the PRRS causing virus diminishes; moreover, the productive parameters of the animals improve. The immunoglobulins obtained can be administered orally and parenterally in aqueous solution.

### BRIEF DESCRIPTION OF THE DRAWINGS

FIG. 1. Shows the determination of antibodies in the serum of pigs treated with two different doses of immunoglobulins administered intramuscularly.

FIG. 2. Shows the results of presence of antibodies against PRRS measured by the ELISA test in treated and control sows.

### DETAILED DESCRIPTION OF THE INVENTION

The detailed characteristics of this novel invention are described hereinafter.

The present invention is based on the fact that the immunoglobulins extracted of the aqueous phase of the egg yolk laid by immunized hens grant protection against viral and bacterial illnesses.

A main mistake of people that work in this field is to use a commercially available virus, however, due to their mutation ability it is very likely that vaccines or passive immunization schemes would fail. It's also believed that in some cases vaccines are responsible for generating infections.

To obtain effective immunoglobulins against PRRS virus, it's necessary to isolate and grow all viruses in the specific area where protection is sought or where currently infections are occurring. Virus growth is carried out preferably in Marc 145 cells. Each isolated virus or a combination or two or more viruses are used to make one or more water in oil vaccines, but any type of vaccine could be used in the practice of the present invention.

The vaccines are then used to induce immunization in layer hens by means well know to a person skilled in the art and eggs from the time they are laid until 30 weeks later are collected.

The vaccination schedule was made in the following way: a 0.3-0.8 ml dose consisting of water in oil emulsion vaccine (70% oil and 30% water) containing one or more isolated PRRS viruses previously inactivated with 0.1% Formol was administered subcutaneously to the hens, 8 weeks of age, in the middle posterior third part of the neck. The complete vaccination program included 2 or more booster administrations, at a 4-8 weeks interval during the laying period.

There are different methods to extract Igs from egg yolk such as the used by Yokoyama (Yokoyama, H. et al 1993) with the modification that Avid AL was not used. The extraction of antibodies from the yolk was made in two steps. In the first step, the yolk was diluted 1:4 (without albumin) with 0.01% sodium azide and stored under refrigeration during at least 24 hours. Then, the supernatant was separated and then 5% hydroxypropylmethylcellulose phtalate (HPMCP) was added in the proportion of 0.25 ml for every 100 ml of yolk. It was allowed to rest for at least 24 hours. The lipid layer that formed in the upper part of the solution was separated to obtain the anti PRSSV immunoglobulin yolk. It was filtered and bottled. The quality control tests include:
1. Sterility test (to check that the product is free from bacterial, fungi and yeast contamination according to the Code of Federal Regulations of the United States of America.
2. Quantification of antibodies against PRRS. The technique of micro virus serum neutralization, beta method (dilution of constant virus sample) is used, on 96-well microplates, flat bottom and MA104 cell growth. The immunoglobulins are diluted from 1:40 to 1:10240 on the microplate using medium 199 as diluent, 200 DICT 50 (infective dose in tissue culture) of PRRS virus are added, incubation at 37° C. during 30 minutes and transfer of the mixture to a monolayer of MA1104 cells of 24 hours of incubation, then incubation during 4-5 days at 37° C. and 5% CO₂. A titer of more than 160 is considered satisfactory to be able as PRSSV neutralizing immunoglobulins.

The following step is to select one or more anti-PRRSV immunoglobulins that neutralize viruses found in a farm by isolation. This is made by conducting neutralization tests on all viruses isolated from the farm. A preferred embodiment of the invention is to mix 2 or 5 neutralizing PRRSV immunoglobulins obtained from 2 or 5 different strains of PRRS virus to ensure the neutralization of all virus believed to be found in a farm.

Another embodiment of the invention is obtaining anti-PRSSV immunoglobulins yolk from hens with a vaccine consisting of a mix of strains of PRSS virus that its anti-PRSSV immunoglobulins neutralizes in combination all viruses identified in the isolation step.

It was found that in some cases a single antiPRRSV immunoglobulin with a titer of 1:10240 neutralizes more than 30 strains of PRRS virus. The criteria to select a virus to be added to the vaccine used to induce and obtain immunoglobulins or to add its immunoglobulins to the anti-PRSSV composition is that the virus should produce immunoglobulins with a titer of more than 1:160, measured by microneutralization tests, and in combination all virus should be neutralized.

An anti-PRRSV composition is produced by mixing the resulted anti-PRSSV immunoglobulins from egg yolk in equal ratio to the quantity of virus neutralized by each specific anti-PRRSV immunoglobulin, produced on a one by one basis, adding water and a suitable preservative, such as sodium azide, at a necessary rate to obtain the following specifications: anti-PRRSV immunoglobulins yolk 15-30%, water 70-85% and preservative 0.001-0.03%.

The composition is suitable for both PRSSV infection prevention and therapeutic actions. The anti-PRSSV composition could be administered orally as a preventive method in neonatal pig during its first 12 hours of life in a 3-7 ml. This oral administration increased protection according to PCR and ELISA test carried out by the applicants, not mentioned in this disclosure. Administration of anti-PRSSV composition is made by parenteral route every 2-4 weeks at a rate of 2-5 ml for young animals (aged 2 to 10 weeks or with weight less than 40 kg) and at rate of 7-11 ml for adult animals (with weight more than 40 kg). For treatment purposes, the administration rate of anti-PRSSV immunoglobulins could be twice the prevention dose.

The following tests are presented as non-limitative examples. Said tests show the use of immunoglobulins against PRRSV in piglets object of the present invention.

### EXAMPLE 1

41 PRRS virus were isolated and identified from an infected farm by growth in Marc 145 cells. A water in oil 70-30% vaccine type from each virus was made and a flock of layer hens for each vaccine were vaccinated according to the invention describe above. After four weeks post vaccination all eggs from hens was collected to obtain serum and egg yolk. and determined titer by virus neutralization test, founding a specific immunoglobulin with a titer of 1:10240. With these specific immunoglobulins a PRSSV neutralization test with all virus isolated from farm was conducted. Results showed that specific anti-PRRSV immunoglobulin neutralized about 90% of total virus isolated. In this case only the 3 field PRRS viruses numbered 6, 10 and 27 from the table shown below did not show any measurable neutralization.

In order to have effective prevention and treatment action, specific antiPRRSV immunoglobulins obtained from these three specific PRRSV were added to previously selected anti-PRSSV immunoglobulins.

| Isolate No | Titer |
|---|---|
| 1 | 5120 |
| 2 | 640 |
| 3 | 5120 |
| 4 | 320 |
| 5 | 160 |
| 6 | <160 |
| 7 | 5120 |
| 8 | 320 |
| 9 | 10240 |
| 10 | <160 |
| 11 | 160 |
| 12 | 10240 |
| 13 | 320 |
| 14 | 160 |
| 15 | 320 |
| 16 | 320 |
| 17 | 160 |
| 18 | 320 |
| 19 | 10240 |
| 20 | 2560 |
| 21 | 2560 |
| 22 | 640 |
| 23 | 160 |
| 24 | 320 |
| 25 | 320 |
| 26 | 320 |
| 27 | <160 |
| 28 | 2560 |
| 29 | 2560 |
| 30 | 160 |
| 31 | 320 |
| 32 | 160 |
| 33 | 160 |
| 34 | 160 |
| 35 | 640 |
| 36 | 1280 |
| 37 | 160 |
| 38 | 160 |
| 39 | 160 |
| 40 | 160 |
| 41 | 20480 |

### EXAMPLE 2

Three sows, 50 days of age, weighing about 20 kg, were placed in 2×2 m pens, and individually identified. One of them received a 5 ml dose of the Ig against PRRS composition of Example 1 equivalent to a 0.4 ml per kg of body weight dose, intramuscularly. Another sow received twice the dose of same Ig against PRRS composition (10 ml) through the same route. The third sow is a control animal, without treatment. Before the immunoglobulin application, a blood sample was taken from the three sows to determine the presence of antibodies against PRRS through the MNT test for PRRS. During 4 weeks after the treatment, blood samples were taken from the sows to determine the antibody levels caused by the antiPRSSV immunoglobulins through the MNT test against PRRS in MA 104 cells. The lesions at the site of application were also assessed as well as any sign suggesting the presence of the illness.

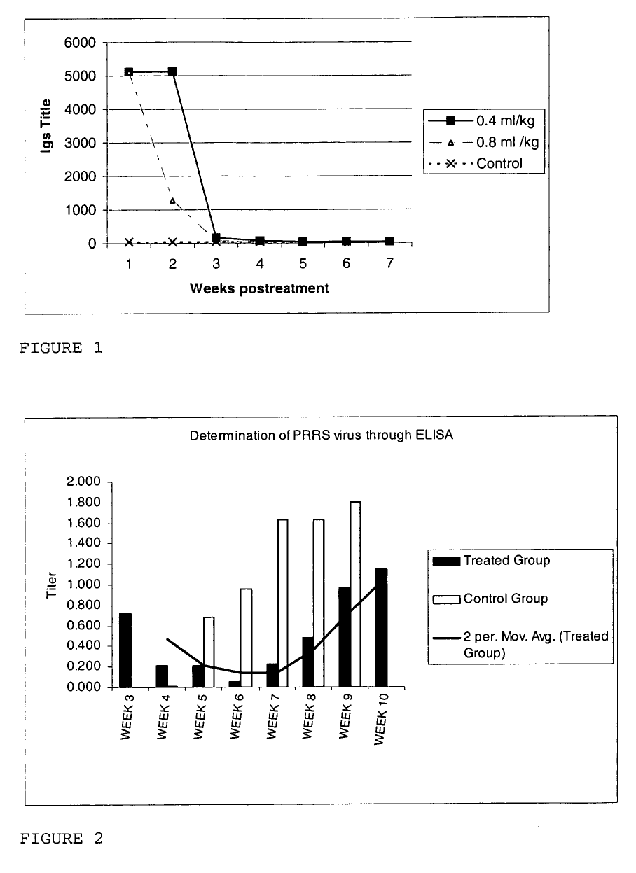

FIG. 1 presents the results obtained in the immunized sows. It can be seen that with the two treatments using immunoglobulins, high levels of antibodies against PRRS were obtained in the first week after the treatment and then a notable diminution is seen, but the levels are still higher than in the control sow. This is an indication of the half life and shows that the antibodies supplied by the present invention remain in the blood flow during three weeks.

### EXAMPLE 3

Four hundred and fifty-two piglets weighing about 7 kg were administered anti PRSSV Igs composition at 10 ml dose intramuscularly, repeating the dose two weeks after the first administration. On the other hand, 420 control piglets were not administered a treatment. The evaluated parameters were weight gain, the virus presence through PCR and ELISA test for PRRS and mortality percentage.

The following results show the parameters of weight gain and mortality in both groups.

| | Treated Group | Control Group |
|---|---|---|
| Number of Animals | 452 | 420 |
| Initial weight | 6.17 | 6.05 |
| Final weight | 26.55 | 30.55 |
| Mortality | 19 | 53 |
| Mortality % | 4.20 | 12.62 |

It was observed that treated group showed lower weight gain compared to the control group. Mortality percentage was reduced 64% in the group treated with immunoglobulins compared to the control group. In the same way, the PCR test was positive in the control group from the fourth week on, while in the group treated with immunoglobulins, a positive result was seen starting at the ninth week after the immunoglobulin treatment.

FIG. 2 presents the results of the ELISA test with serums of treated and controlled pigs. The results show a lower exposition of the pigs to the infectious agent in the group treated with immunoglobulins compared to the control group, in which the presence of the virus was detected after the fifth week. FIG. 2 also shows the mortality of the treated pigs, the serology obtained and the weight gain.

### EXAMPLE 4

10 pigs divided in 2 groups of 5 pigs of 21 days old were maintained in isolation units under negative pression. All animals were from a negative PRRS farm. A sample of serum was taken to carry out PCR simple and nested tests to establish the negative condition of the pigs. All pigs were inoculated with 1 ml of a field strain PRRSV (10^{4.0} TCID/ml) by intramuscular route. All animals received food and water at liberty. After 2 days postchallenge each pig of one group were treated with a dose of 0.8 ml per kg body weight of anti-PRSSV composition of Example 1. A sample of serum was taken at 3, 7, 14, 21 and 28 days after treatment with Igs—antiPRRS. Serum from blood were separate and PCR and ELISA tests were run.

Results are shown in the next table.

| | D.P.C. | | | | | | | | | | | |
|---|---|---|---|---|---|---|---|---|---|---|---|---|
| | 2 | | 5 | | 9 | | 16 | | 23 | | 30 | |
| | D.P.T. | | | | | | | | | | | |
| | 0 | | 3 | | 7 | | 14 | | 21 | | 28 | |
| Treated | PCR | 0+/5 | PCR | 0+/5 | PCR | 1+/5 | PCR | 0+/5 | PCR | 0+/5 | PCR | 1+/5 |
| group | Elisa | 0+/5 | Elisa | 0+/5 | Elisa | 0+/5 | Elisa | 1+/5 | Elisa | 1+/5 | Elisa | 1+/5 |
| Control | PCR | 0+/5 | PCR | 3+/5 | PCR | 3+/5 | PCR | 1+/5 | PCR | 0+/5 | PCR | 5+/5 |
| Group | Elisa | 0+/5 | Elisa | 0+/5 | Elisa | 0+/5 | Elisa | 2+/5 | Elisa | 3+/5 | Elisa | 4+/5 |

D.P.C. = Days post Challenge
D.P.T. = Days post Treatment

The results shown on the table above indicate that viremia started at 5 Days postchallenge measured by PCR, control group shown a 60% of viremia and treated group shown only 20%. At the end of trial, treated group showed only 20% of viremia versus 100% and 80% of positivity to PRRS measured by PCR and Elisa test.

The above description of certain embodiments are made for the purpose of illustration only and are not intended to be limiting in any manner. Other alterations and modifications of the preferred embodiment will become apparent to those of ordinary skill in the art upon reading this disclosure, and it is intended that the scope of the invention disclosed herein be limited only by the broadest interpretation of the appended claims to which the inventor is legally entitled.

What we claim is:

1. Compositions for prevention and treatment of infections caused by PRRS virus consisting of 15-30% of anti-PRRSV immunoglobulins yolk, 70-85% of water and 0.001-0.03% of a preservative.

2. Compositions for prevention and treatment of infections caused by PRRS virus of claim 1 where anti-PRRSV immunoglobulins yolk consist of one or more specific neutralizing PRRS virus immunoglobulins.

3. Compositions for prevention and treatment of infections caused by PRRS virus of claim 1 where one or more specific neutralizing PRRS virus immunoglobulins are selected in a way that all viruses found in a farm are neutralized.

4. Compositions for prevention and treatment of infections caused by PRRS virus of claim 2 where such specific neutralizing PRRS virus immunoglobulins has a titer greater than 1:160.

5. Compositions for prevention and treatment of infections caused by PRRS virus of claim 1 where such composition is administered to animals by oral route at a dose of 2-7 ml for neonatal pigs.

6. Compositions for prevention and treatment of infections caused by PRRS virus of claim 1 where such composition is administered to animals by parenteral route every 2-4 weeks at a rate of 2-5 ml for pigs under 40 kg.

7. Compositions for prevention and treatment of infections caused by PRRS virus of claim 1 where such composition is administered to animals by parenteral route every 2-4 weeks at dose of 7-11 ml for pigs with weight over 40 kg.

8. Method for prevention and treatment of infections caused by PRRS virus comprising the steps of isolation of all strains of PRRS viruses found in a farm, production of specific neutralizing PRRS virus immunoglobulins, selection of specific neutralizing PRRS virus for all strains of PRRS viruses, production of anti-PRRSV composition, and administration of an anti-PRRSV composition to pigs.

9. Method for prevention and treatment of infections caused by PRRS virus of claim 8 wherein anti-PRRSV composition consists of a suspension containing 15-30% of anti-PRRSV immunoglobulins yolk, 70-85% of water and 0.001-0.03% of a preservative.

10. Method for prevention of infections caused by PRRS virus of claim 9 wherein said anti-PRRSV immunoglobulins yolk contains at least one specific neutralizing PRRS virus immunoglobulins.

11. Method for prevention and treatment of infections caused by PRRS virus of claim 10 wherein said specific neutralizing PRRS virus immunoglobulins neutralizes at least one PRSS virus at a titer at least of 1:160.

12. Method for prevention and treatment of infections caused by PRRS virus of claim 8 wherein administration of anti-PRRSV composition is made orally or intramuscularly.

13. Method for prevention and treatment of infections caused by PRRS virus of claim 8 wherein anti-PRRSV composition is administered to neonatal pigs by oral route at a dose of 2-7 ml.

14. Method for prevention and treatment of infections caused by PRRS virus of claim 8 wherein anti-PRRSV composition is administered to pigs under weight of 40 kg by parenteral route every 2-4 weeks at a rate of 2-5 ml.

15. Method for prevention and treatment of infections caused by PRRS virus of claim 8 wherein anti-PRRSV composition is administered to pigs with weight over 40 kg by parenteral route every 2-4 weeks at dose of 7-11 ml.
